(19) United States
(12) Patent Application Publication
(10) Pub. No.: US 2018/0247710 A1
(43) Pub. Date: Aug. 30, 2018

(54) HEALTH DATA, PLATFORM SYSTEM AND METHOD

(71) Applicants: Anastasia N. Durkin, Kansas City, MO (US); Joseph R. Brisson, Rio Rancho, NM (US)

(72) Inventors: Anastasia N. Durkin, Kansas City, MO (US); Joseph R. Brisson, Rio Rancho, NM (US)

(21) Appl. No.: 15/908,463

(22) Filed: Feb. 28, 2018

Related U.S. Application Data
(60) Provisional application No. 62/464,484, filed on Feb. 28, 2017.

(51) Int. Cl. G16H 40/00; G06F 17/30; G06F 9/54

(52) U.S. Cl. CPC G16H 40/00; G06F 9/445; G06F 9/541; G06F 17/30292

(57) ABSTRACT

A central repository of all health data is accessible through defined application programming interfaces (APIs) to approved service providers. The present invention also accommodates fast healthcare interoperability resources (FHIR) functionality.

### CROSS-REFERENCE TO RELATED APPLICATION

This application claims priority in U.S. Provisional Patent Application No. 62/464,484, filed Feb. 28, 2017, which is incorporated herein by reference.

### BACKGROUND OF THE INVENTION

### 1. Field of the Invention

The present invention relates generally to the transfer, management and access of healthcare data through a series of federated and linked domains. Hospital information systems, and healthcare-related information systems generally, have evolved considerably from simplistic patient registration and billing systems, which handled transactions with insurers and other payers.

The current model for the transfer, management and access of healthcare data utilizes large applications, such as those provided by the Cerner Corporation, EPIC Systems Corporation, or McKesson Corporation. These applications are designed to control, restrict or deter health data from fluid access by third-party solutions.

The present invention allows for a central repository of all health data which is accessible through defined application programming interfaces (APIs) to approved service providers. Data storage is extensively used for health information. The present invention also accommodates Fast Healthcare Interoperability Resources (FHIR) functionality.

### SUMMARY OF THE INVENTION

In the practice of the present invention, a central repository is provided for healthcare-related data, which is accessible through defined application programming interfaces (APIs) to approved service providers. Data storage is an important component of health information management, including the system of the present invention.

### BRIEF DESCRIPTION OF THE DRAWINGS

FIG. 1 shows a prior art hospital information system of the type utilized in the 1980s.

FIG. 2 shows another prior art information system of the type utilized in the 1990s.

FIG. 3 shows yet another prior art hospital information system, including an application centric model, and utilized in about 2000 to 2016.

FIG. 4 shows another prior art hospital information system, including a Fast Healthcare Interoperability Resources (FHIR) interface, and utilized in about 2014 to present.

FIG. 5 shows a healthcare information system utilizing an embodiment of the present invention.

### DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting.

### II. Preferred Embodiment

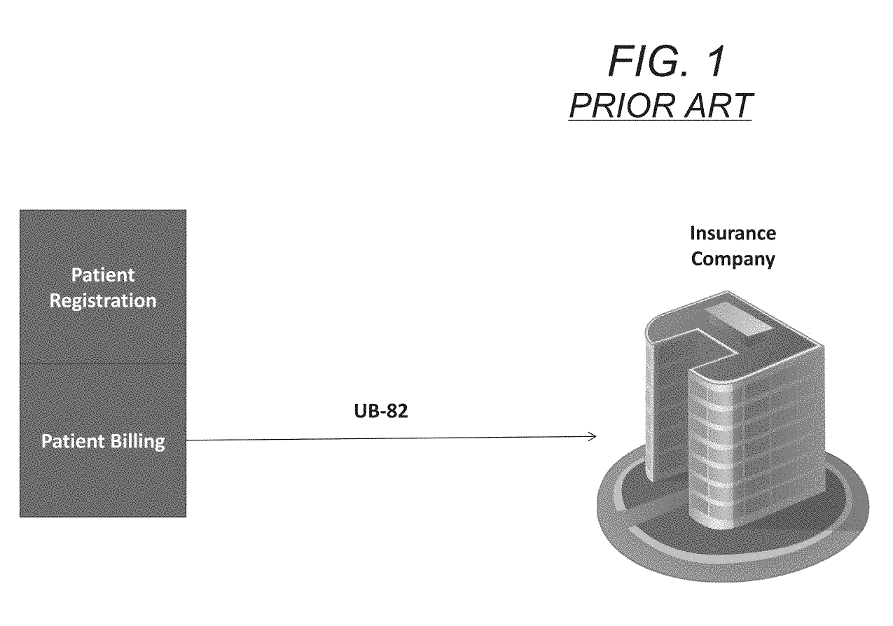

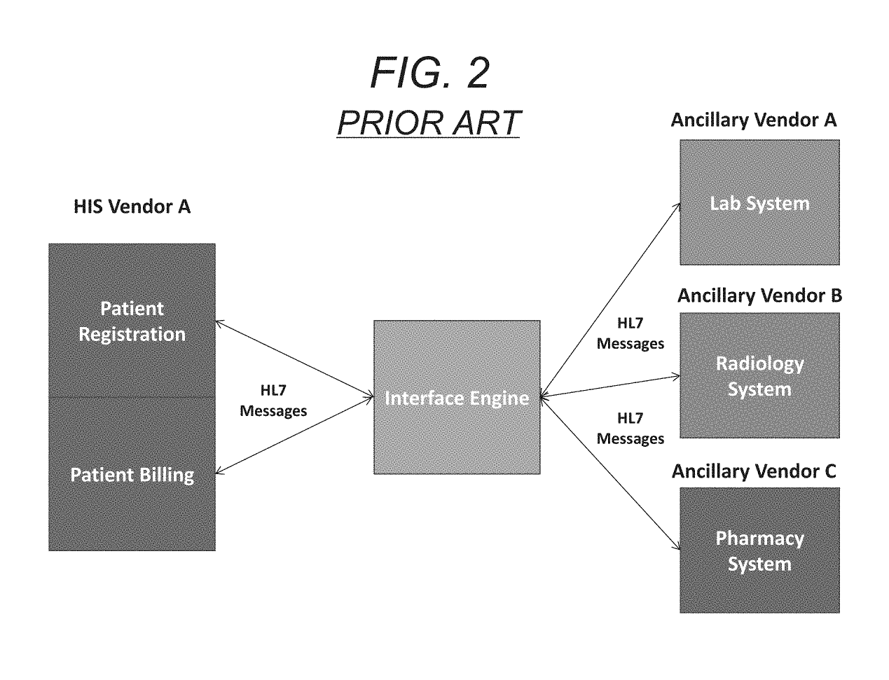

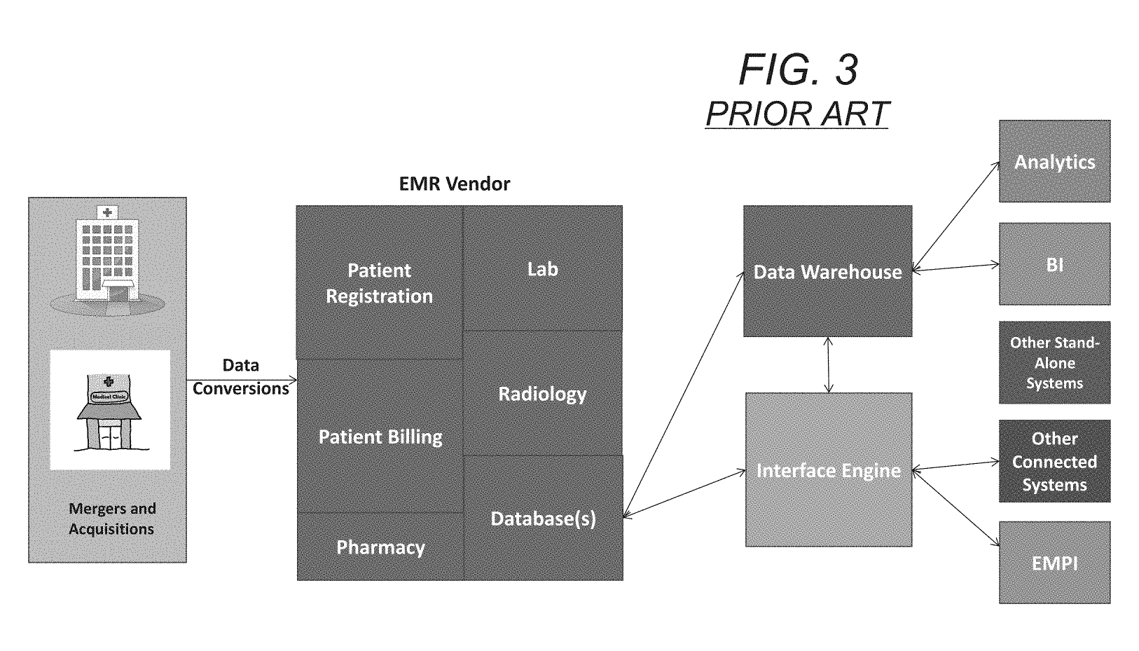

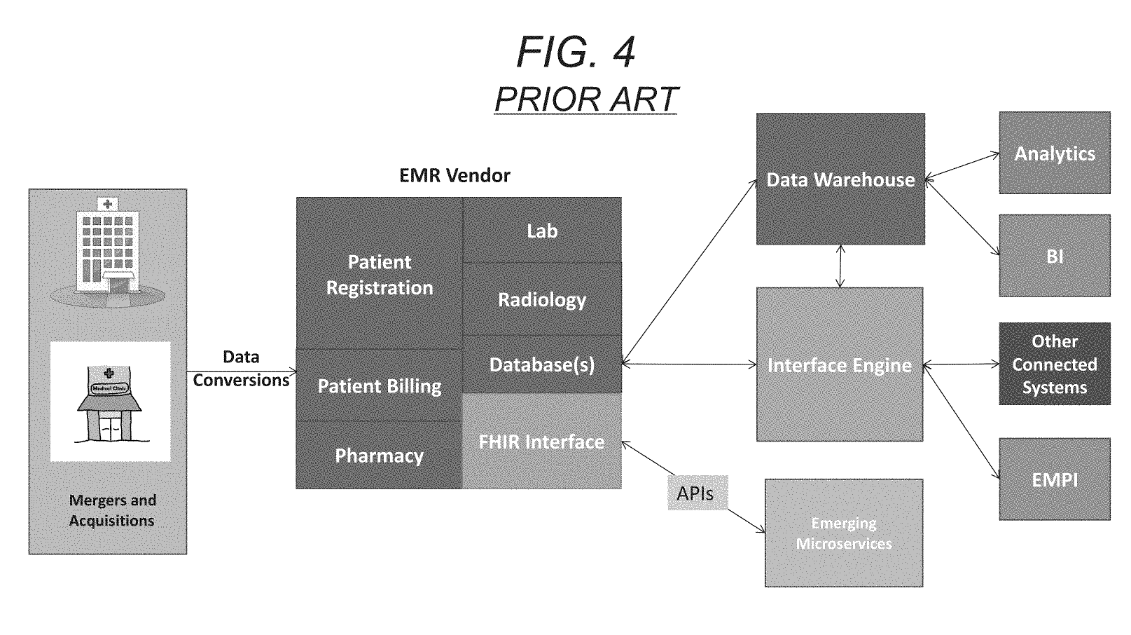

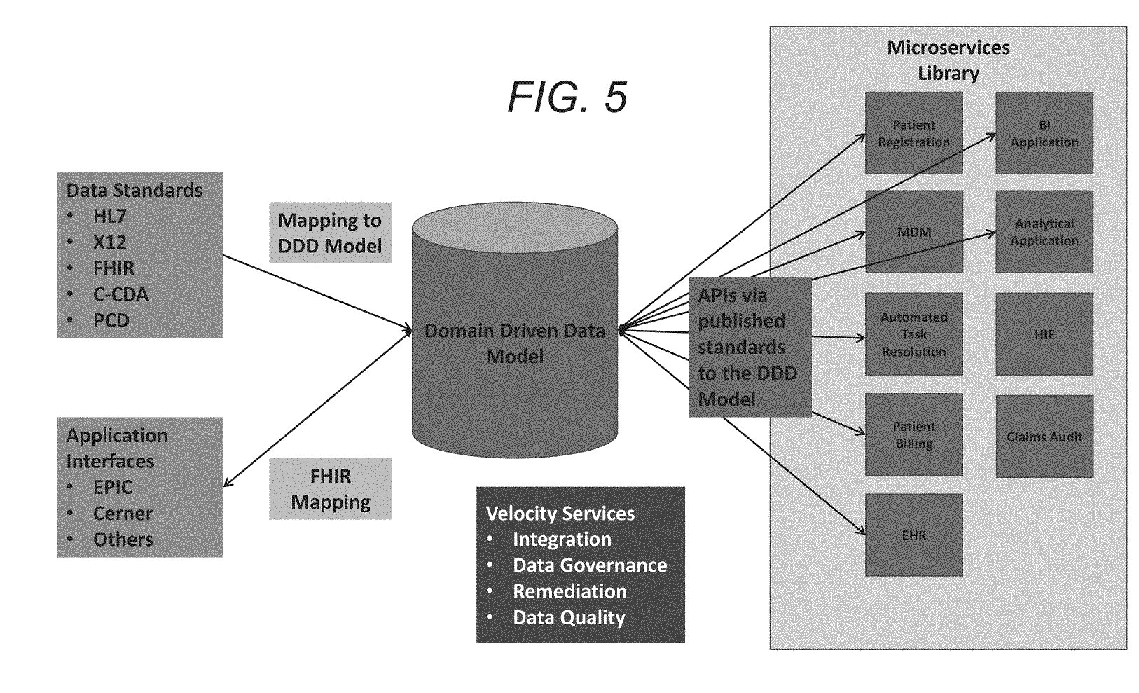

Referring to the drawings in more detail, FIGS. 1-4 show prior art hospital information systems. FIG. 5 shows a healthcare information system embodying an aspect of the present invention, including a data-centric model with an FHIR interface.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. An information management system, which includes:
a domain driven data model connected to data standards and application interfaces;
a microservices library connected to the domain driven data model; and
application programming interfaces (APIs) loaded from said microservices library.
